(19) United States
(12) Patent Application Publication
(10) Pub. No.: US 2021/0030372 A1
(43) Pub. Date: Feb. 4, 2021

(54) METHODS TO ESTIMATE THE BLOOD PRESSURE AND THE ARTERIAL STIFFNESS BASED ON PHOTOPLETHYSMOGRAPHIC (PPG) SIGNALS

(71) Applicant: Evonik Operations GmbH, Essen (DE)

(72) Inventors: Rosario LIZIO, Dieburg (DE); Philipp OCKERMANN, Wuerzburg (DE); Serena MOSCATO, Bologna (IT); Sara LIEBANA VIÑAS, Aschaffenburg (DE); Lorenzo CHIARI, Monte San Pietro, Bologna (IT); Michael HUTH, Maintal (DE)

(73) Assignee: Evonik Operations GmbH, Essen (DE)

(21) Appl. No.: 17/045,910

(22) PCT Filed: Apr. 18, 2019
(86) PCT No.: PCT/EP2019/060146

§ 371 Date: Oct. 7, 2020

(30) Foreign Application Priority Data
Apr. 23, 2018 (EP) 18168758.3

(51) Int. Cl. A61B 5/00; A61B 5/024; A61B 5/021; A61B 5/02; G16H 10/60; G16H 50/30

(52) U.S. Cl. CPC A61B 5/7278; A61B 5/02416; A61B 5/02116; A61B 5/02125; A61B 5/02007; A61B 5/1072; A61B 5/681; A61B 5/7239; G16H 10/60; G16H 50/30; A61B 5/7203; A61B 5/02405

(57) ABSTRACT

A method can estimate the blood pressure and the arterial stiffness based on photoplethysmographic (PPG) signals. Algorithms can be based on PPG signals to analyze the cardiovascular condition of a person by estimating cardiovascular parameters. A method can measure one or more cardiovascular parameters in a subject based on PPG signals.

The present invention relates to a method to estimate the blood pressure and the arterial stiffness based on photoplethysmographic (PPG) signals. New algorithms have been developed and validated based on PPG signals to analyze the cardiovascular condition of a person by estimating cardiovascular parameters. With the present invention a method for measuring one or more cardiovascular parameters in a subject based on PPG signals is provided.

Photoplethysmographic (PPG) sensors can be found in a number of different devices. Not only are they built into consumer goods such as wrist-type fitness trackers but also into devices used by medical professionals. The sensors are mostly used to either estimate the pulse rate or the oxygen saturation in the blood.

A plethysmograph is an instrument that measures changes in volume of an organ and is basically an optical sensor. The term photoplethysmography usually refers to the measurement of volume changes in arteries and arterioles due to blood flow. There are different kinds of PPG sensors.

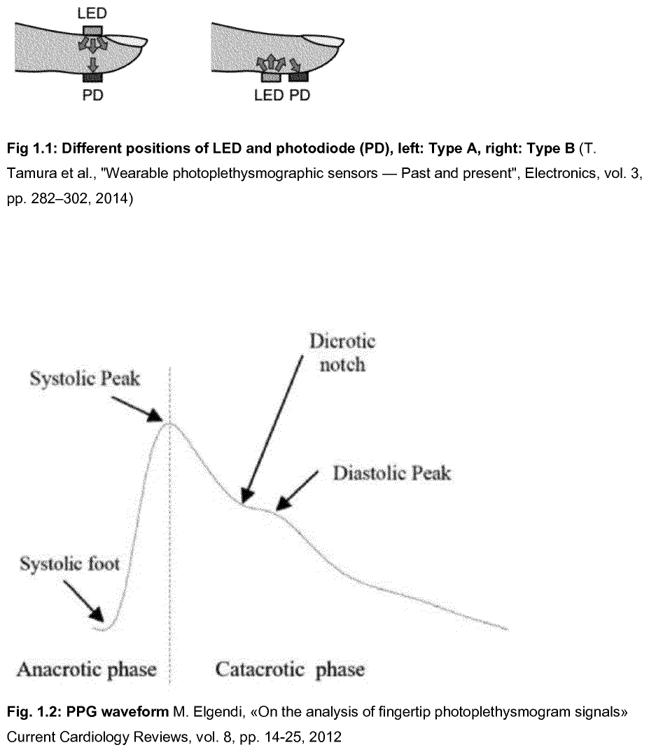

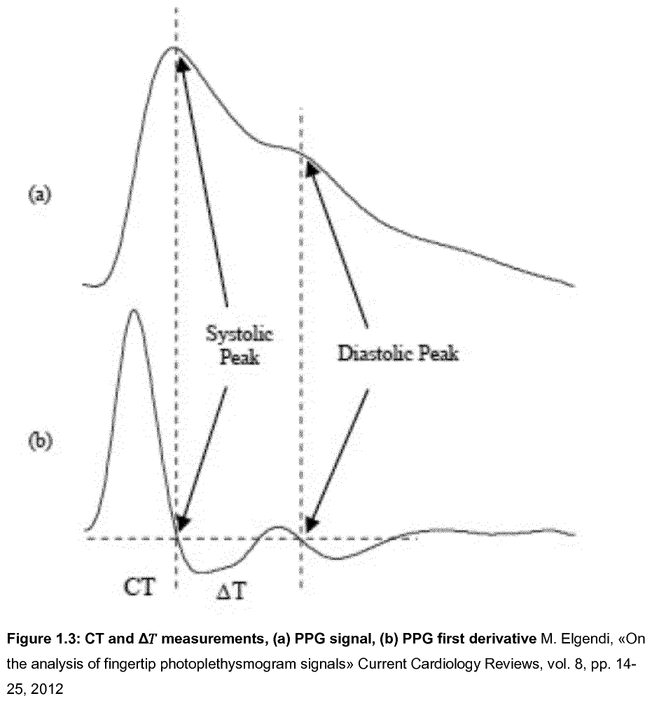

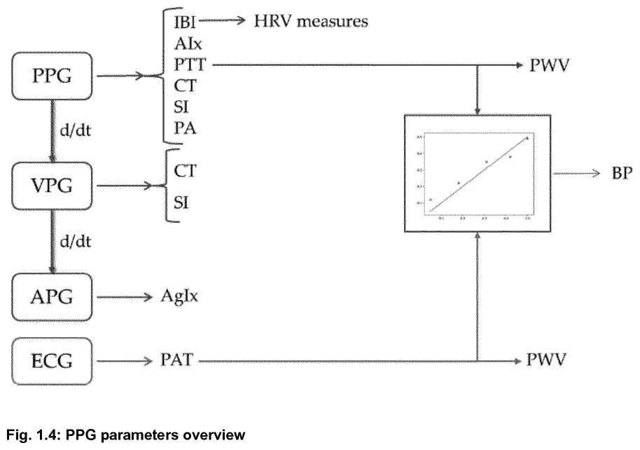

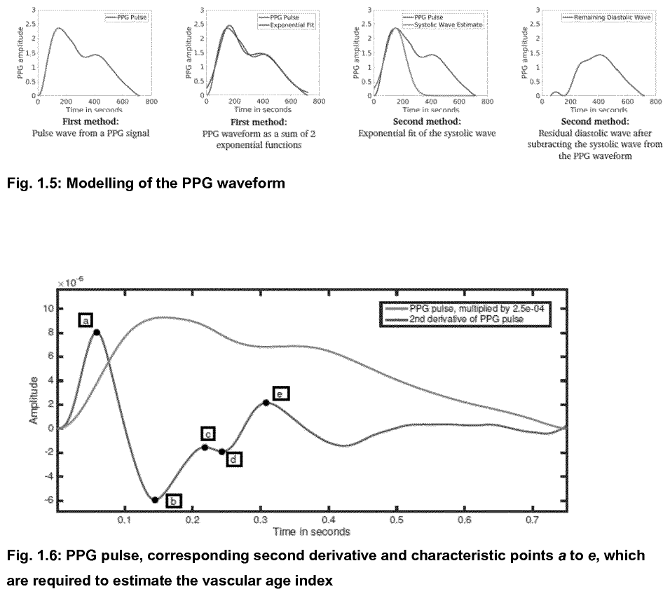

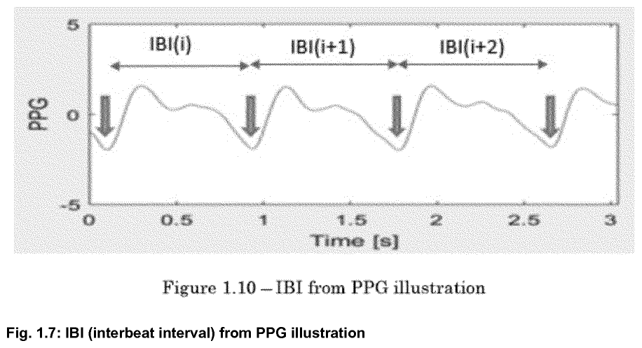

Some are placed at the fingertip, some at the wrist and other sites such as the ear lobe are also possible. The sensor itself consists of a light emitting diode (LED) that emits light onto the skin and of a photodiode. This diode is usually placed next to the LED, detecting light that is reflected (Type B). For finger sensors, the photodiode can also be placed at the opposite end of the finger, measuring the light that travels through the finger (Type A). FIG. 1.1 shows the different types.

The PPG sensor placement can affect the signal quality and the robustness to motion artifact. Light wavelength, configuration, and the successive analysis depend on the measurement site (Castaneda et al., International journal of biosensor & bioelectronics, vol. 4, n. 4, pp. 195-202, 2018). The light wavelength is a relevant project issue (which also affects the photodetector system). Generally, a PPG device works at red or near-IR wavelength. Thanks to its optical features, this kind of light source provides excellent deep-tissue (e.g. into muscles) blood flow measurements. Recently, more and more commercial sensors are equipped with a green light source: it is suitable for superficial measures (e.g. arterioles), it provides a larger signal modulation (Tamura et al., Electronics, vol. 3, pp. 282-302, 2014) and it has a better signal-to-noise ratio than the IR source (Jing et al., 38th Annual International Conference of the IEEE Engineering in medicine and biology society, 2016).

### PPG Waveform

Based on the different layers in which the light propagates, the PPG waveform comprises two parts: a pulsatile (AC) physiological waveform, attributed to cardiac synchronous changes in the blood volume (in vessels) with each heartbeat, which is superimposed on a slowly varying (DC) component. DC or static signals are determined by static elements of body tissue such as, for instance, epidermis, bones and non-pulsatile blood.

The photoplethysmography signal within a cardiac cycle has a stereotyped waveform. Two phases can be detected: the anacrotic phase and the catacrotic phase. The former is mainly due to the systolic event of the cardiac cycle, the latter is partially caused by the diastolic event and by the reflection of the pressure wave by the peripheral vessels.

Landmark points can be detected within the PPG waveform as shown in FIG. 1.2. The systolic foot is defined as the minimum value of the PPG wave during the cardiac cycle. The systolic peak is the maximum point. Both points fall in the anacrotic phase. The diastolic peak is the second maximum. The dicrotic notch is a slight negative inflection between the systolic peak and the diastolic peak; whether this notch is present or not depends on several factors (such as age or measurement site). Both the dicrotic notch and the diastolic peak fall in the catacrotic phase.

### Sensor Placement

The PPG sensor placement can affect the signal quality and the robustness to motion artifact. Light wavelength, configuration, and the successive analysis depend on the measurement site. The most common measurement site is the fingertip: it is used in the intensive care units in order to obtain information about oxygen saturation (commonly, it is called “pulse oximeter”). Thanks to the large signal amplitude that can be achieved compared to other measurement sites, this measurement can be considered the gold standard for the PPG signal. However, the greatest disadvantage of this site is that this kind of sensor interferes with daily activities, so it is not suitable for pervasive sensing.

Recently, many research groups have focused on wrist PPG measurement. Unfortunately, high performance cannot be obtained in this site because of motion artifacts, which do not still allow to achieve high reliability. There are several studies about the differences in PPG signal comparing different measurement sites, for example the fingertip, the wrist, the earlobe, the forehead, and the toe. In a recent study (Rajala et al., Physiological measurement, vol. 39, p. 13 pp, 2018), PPG signals recorded from the wrist and the fingertip were compared. Results show that the wrist PPG waves are different from the fingertip ones both in shape and amplitude. Nevertheless, authors affirm that the wrist PPG signal can be used for several cardiovascular parameter estimates that can provide useful information about the blood pressure. Another recent article (Han and Shin, World Congress on Medical Physics and Biomedical Engineering, 2018) discloses a study to assess the best measuring position on the wrist and the optimal wavelength to record the PPG signal, considering the fingertip as the gold standard. As a result, they found the dorsal radial artery and the green light source as best measuring position and wavelength.

A plethysmographic measurement can provide several parameters and indicators, thanks to which it's possible to obtain information about the cardiovascular system. The continuous research for new parameters is driven by the high portability of a photopletysmographic system: the classical measurement technique, which often involves bulky instrument, could be replaced with this kind of instrument, that is easy to set up and also allows continuous monitoring.

### Relation Between Cardiovascular Parameters and Arterial Stiffness

With increasing age, the blood vessels usually become stiffer compared to those of a young person. This phenomenon occurs primarily because elastin in blood vessels' walls deteriorates and is replaced by collagen, which is less flexible. The increased stiffness causes the blood to travel faster through the vessels, therefore arterial stiffness is strongly correlated to the pulse wave velocity PWV. If a person's arterial stiffness is higher than the normal value for their age, this is a determinant of hypertension, i.e. increased systolic and diastolic blood pressure. As mentioned above, hypertension is an increasingly large problem, thus arterial stiffness is of interest as well. Since increased arterial stiffness can be detected before hypertension occurs, this allows to start treatment or behavioral changes early, possibly avoiding hypertension. It is also well known that atherosclerotic plaques and aneurysms involve changes in vessel wall properties and therefore their stiffness (M. McGarry et al., “In vivo repeatability of the pulse wave inverse problem in human carotid arteries”, J. of biomechanics, vol. 64, pp. 136-144, 2017). Also in this case, an accurate arterial stiffness measurement, in particular its variation, would improve diagnosis and monitoring of the connected diseases. Various cardiovascular parameters can be analyzed to gain information about a person's cardiovascular health.

Augmentation index (AIx) is a cardiovascular parameter that is usually obtained from a pressure pulse wave and can be measured at a large artery with a device that uses an inflatable cuff. In contrast, the PPG sensor is unable to measure pressure and only detects volume changes in very small arteries and arterioles. It provides an indirect measure of arterial stiffness and further provides information about the pressure wave reflection by the peripheral circulatory system. The Augmentation Index measure was transposed from the Blood Pressure Pulse Wave Analysis to the PPG signal, assuming that one is able to obtain information about the arterial stiffness analyzing the PPG waveform. Just like arterial stiffness, the augmentation index increases with age and can be used to estimate the risk of suffering from a cardiovascular disease in the future.

Vascular age index (AgIx) is a cardiovascular parameter that gives information on the age condition of the arteries, compared to some normal threshold for a healthy population. It can be determined with devices that uses an inflatable cuff. In the literature the AgIx as given from the second derivative of the PPG pulse wave form. The vascular age is mainly influenced by a genetic predisposition and by the lifestyle. The estimate of this parameter is based on the pressure wave velocity through the vascular tree. In healthy subjects, it should be lower than the chronological age. In hypertensive subjects, it is significantly higher than the chronological age (Lozinsky, Arterial Hypertension, vol. 19, n. 4, pp. 174-178, 2015).

Pulse wave velocity (PWV) describes the velocity of blood that travels through a person's arteries and is used as a measure of arterial stiffness. PWV is defined as the speed at which the pressure wave propagates through the cardiovascular tree. The PWV assessment provides information about the elastic properties of the arterial system. The most precise devices to measure PWV perform a carotid-femoral measurement. For this measurement, one tonometer is placed at the carotid artery which is located at the neck and a second tonometer is placed at the femoral artery at the upper leg. Those tonometers measure the pressure pulse waves of the arteries. From the time difference between the signals and the distance between the tonometers, PWV can be calculated. A more convenient way to estimate the PWV is by using two PPG sensors at a known distance or one PPG sensor and an electrocardiogram (ECG) and to calculate PWV from the time difference between the signals. Although it is more difficult to assess, the pulse transit time (PTT) provides a better measure for monitoring. This parameter would allow estimating the aortic PWV (the aorta is the reference point to measure the PWV in the literature). PWV can also be measured with only one blood pressure cuff. This technique is used by the “Mobil-OGraph PWA” which is a clinical device by I.E.M. GmbH that has been used as a reference device in the experimental setup.

Blood pressure (BP) denotes the pressure that the blood traveling through a large artery exerts onto its walls. Hypertension is a major risk factor for multiple diseases, such as stroke and end-stage renal disease, and overall mortality. By the year 2025, it is expected that the number of people across the world who are hypertensive will have risen to 1.56 billion. If the condition is detected early and treated properly, the risk of disease can be decreased significantly. Therefore, it is important to measure BP regularly in order to detect abnormal changes. Besides this, a change of lifestyle can often decrease BP and prevent hypertension, provided that a tendency towards it is detected early. Currently, there exist several different approaches to measure BP. The most common device is an inflatable cuff that is placed at the patient's arm and that applies pressure onto the brachial artery. While this allows an accurate measurement, it is perceived as inconvenient by some patients and it requires a visit to a doctor or the purchase of a device. Other approaches are invasive, such as intravenous cannula that are placed inside an artery. Those are only used in a clinical context, e.g. during a surgery. A PPG signal can be obtained comfortably, continuously and at low cost. Extracting information about BP can serve an important purpose: As it is easy to obtain at home, this could warn a person early and advise them to seek medical advice.

Heart rate variability (HRV) describes the variation in the time interval between heartbeats and is usually calculated from an ECG, as the RR intervals from the ECG are required. Nevertheless, for the HRV analysis, in principle, any signal that allows accurately identifying heartbeats can be used.

For this reason, the PPG technology seems to be a valid alternative for conducting an HRV analysis (Pinheiro et al., IEEE Explore Digital Library, 2016). Normally, the HRV is determined from the PPG signal based on determining the locations of the systolic feet.

### Other PPG Parameters

In addition to the aforementioned parameters, various morphological characteristics of the PPG signal and its derivatives have also been studied.

The Pulse Area is defined as the area under the PPG curve. In a recent study (Usman et al., Acta Scientiarum Technology, vol. 36, n. 1, pp. 123-128, 2013), a significant difference in this parameter was found in relation to two different levels of diabetes. In conclusion, the authors affirmed that it can be used as a useful parameter in determining arterial stiffness. In the work of Wang et al. (Annual International Conferente of the IEEE Engineering in Medicine and Biology Society, 2009), the area is divided into two sub-areas, A1 and A2, at the dicrotic notch. Based on these two measures, the Inflection Point Ratio was defined as the ratio between the two areas, demonstrating that this ratio can be used as an indicator of total peripheral resistance.

The time ΔT between the systolic peak and the diastolic peak seems to be linked to the blood vessels elasticity. Millasseau et al. (Clinical Science, vol. 103, n. 4, pp. 371-377, 2002) used this time interval to obtain a new index, the Large Artery Stiffness Index (SI), defined as the ratio between the height of the subject and the time interval between the systolic and diastolic peaks, finding that it decreases with age.

Another measure of the PPG signal temporal trend is the Crest Time (CT). Easy to measure, the CT is the time elapsed between the systolic foot and the systolic peak of a PPG wave. It has been assessed as a valid parameter (together with other measurements deriving from the PPG signal) for a cheap and effective Cardiovascular Disease (CVD) screening technique for use in general clinical practice (Alty et al., IEEE Transactions on biomedical engineering, vol. 54, n. 12, pp. 2268-2275, 2007).

The CT and the SI can be estimated in a more reliable way using the first derivative of the PPG signal, also known as Velocity Photoplethysmograph (VPG), measuring the time interval between the relative zero-cross (see FIG. 1.3).

FIG. 1.4 presents a graphical summary of the parameters described above that can be obtained from the study of the PGG signal.

Different systems for measuring blood pressure as an alternative to inflatable cuffs have been described, such as in WO 2015/066445 A1 where a system and method for measuring and monitoring blood pressure is provided. The system includes a wearable device and a tonometry device coupled to the wearable device. The Tonometry device is configured to compress a superficial temporal artery (STA) of a user. A sensor pad is attached to the wearable device adjacent the tonometry device. A blood pressure sensor is integrated within the sensor pad for continuous, unobtrusive blood pressure monitoring.

WO 2015/193917 A2 discloses a method and system for cuff-less blood pressure (BP) measurement of a subject. The method includes measuring, by one or more sensors, a local pulse wave velocity (PWV) and/or blood pulse waveforms of an arterial wall of the subject. Further, the method includes measuring, by an ultrasound transducer, a change in arterial dimensions over a cardiac cycle of the arterial wall of the subject. The arterial dimensions include an arterial distension and an end-diastolic diameter. Furthermore, the method includes measuring, by a controller unit, BP of the subject based on the local PWV and the change in arterial dimensions.

Further, different approaches for measuring one or more cardiovascular parameters have been proposed. US 201600089081 A1 describes a wearable sensing band that generally provides a non-intrusive way to measure a person's cardiovascular vital signs including pulse transit time and pulse wave velocity. The band includes a strap with one or more primary electrocardiography (ECG) electrodes which are in contact with a first portion of the user's body, one or more secondary ECG electrodes, and one or more pulse pressure wave arrival (PPWA) sensors. The primary and secondary ECG electrodes detect an ECG signal whenever the secondary ECG electrodes make electrical contact with the second portion of the user's body, and the PPWA sensors sense an arrival of a pulse pressure wave to the first portion of the user's body from the user's heart. The ECG signal and PPWA sensor(s) readings are used to compute at least one of a pulse transit time (PTT) or a pulse wave velocity (PWV) of the user.

The use of PPT for analyzing cardiovascular parameters has been described in the state of the art, such as in US 2015/0148663 A1 proposing a photoplethysmographic measurement apparatus, a photoplethysmographic measurement method, and an apparatus for measuring a biosignal. The photoplethysmographic measurement apparatus includes a probe, a light emitter comprising a nonelectrical light source and disposed at one end of the probe, the light emitter configured to illuminate a measurement part, and a light receiver disposed at another end of the probe and configured to detect light reflected or transmitted by the illuminated measurement part.

In WO 2014/022906 A1 a system is provided that continuously monitors cardiovascular health using an electrocardiography (ECG) source synchronized to an optical (PPG) source, without requiring invasive techniques or ongoing, large-scale external scanning procedures. The system includes an ECG signal source with electrodes contacting the skin, which generates a first set of information, and a mobile device having a camera which acts as a PPG signal source that generates a second set of information. Together with the mobile device's processor, configured to receive and process the first and second sets of information, from which the time differential of the heart beat pulmonary pressure wave can be calculated, continuous data related to cardiovascular health markers such as arterial stiffness can be determined. Variations of the ECG source may include a chest strap, a plug-in adaptor for the mobile device, or electrodes built into the mobile device.

US 2013/324859 A1 discloses a method for providing information for diagnosing arterial stiffness noninvasively using PPG. The method of the invention for assessing arterial stiffness comprises: a user information input step, characteristic point extraction step, and arterial stiffness assessment step. In particular the arterial stiffness assessment step includes the result of performing multiple linear regression analysis using the baPWV (branchial-ankle pulse wave velocity) value. PPG segmentation is conducted with the help of the PPG second derivative and the PPG pulses need to be classified to remove corrupted PPG pulses. The additional cardiovascular features, such as augmentation index and vascular age index are directly estimated from the characteristic points of the second derivative waveform. Moreover, the second derivative is used to find the position in the PPG signal of some pivotal points.

The US 2017/0238818 A1 describes a method for measuring blood pressure including illuminating by one PPG sensor included in an electronic device, the skin of a user and measuring a PPG signal based on an illumination absorption by the skin. The method also includes extracting a plurality of parameters from the PPG signal, wherein the parameters may comprise PPG features, heart rate variability (HRV) features, and non-linear features.

Elgendi (Current Cardiology Reviews, 2012, 8, 14-25) describes the use of PPG to estimate the skin blood flow using infrared light. Recent studies emphasize the potential information embedded in the PPG waveform signal and it deserves further attention for its possible applications beyond pulse oximetry and heart-rate calculation. Especially, characteristics of the PPG waveform and its derivatives may serve as a basis for evaluating vascular stiffness and aging indices.

The European patent application EP 3061392 A1 discloses a method for determining blood pressure comprising means for providing pulse wave data representative of the heartbeat of a human subject, which has a body height, an age and a gender. The blood pressure of the subject is determined based on the time difference between two peaks in the same PPG pulse, the body height, age and gender.

However, all these solutions require different sensors and are not adapted to be implemented in a compact wrist-worn device. Besides, all these methods do not include individual physiological parameters of the measured subject, but only rely on the measured values.

Therefore, proceeding from the prior art, there is a need for a method to estimate the blood pressure and the arterial stiffness based on PPG signals and provide optimized algorithms for the calculation of different cardiovascular parameters based on individual physiological parameters of interest, such as height, age and other estimated parameters, such as the heart-rate. It is desired to provide a multi-functional solution that incorporates as much parameters as possible. The proposed solution should be incorporated into a compact system, such as a wrist-band or smartwatch, where additional functionalities related to the monitoring of various cardiovascular parameters could be included.

The problem is solved by providing a method for measuring one or more cardiovascular parameters in a subject, by estimating one or more cardiovascular parameters in a subject, the subject having an age and a body height with the following steps:
determining the age (page) and body height (pheight) of the subject,
measuring at least two photoplethysmographic (PPG) signals with at least two PPG sensors at two different positions at the subject,
separating the PPG signal into PPG pulses, whereby the start point and the end point of the pulse corresponds the systolic foot of the PPG signal,
determining the heart rate of the subject (pHR) and calculating the median heart rate,
determining the systolic Asys and diastolic Adia peak amplitudes and their times ts and td,
calculating the second derivative of the PPG pulse, and determining the characteristic points a, b, c, d, and e from the second derivative of the PPG pulse, wherein
   a and e are the first and second most prominent maxima in the second derivative, respectively,
   c is the most prominent peak between the points a and e,
   b is the most prominent minimum in the second derivative and,
   d is the most prominent minimum between points c and e,
determining:
a) the vascular age index AgIx using linear regression based on the characteristic points a, b, c, d, and e, age (page), body height (pheight) and median heart rate of the subject,
b) the pulse wave velocity PWV using linear regression based on the time difference between the two PPG pulses (PTT), age (page), body height (pheight) and median heart rate estimation of the subject,
c) blood pressure BPdia and BPsys using linear regression based on time difference between the two PPG pulses (PTT) and median heart rate and
d) optionally the augmentation index AIx, based on the systolic Asys and diastolic Adia peak amplitudes normalized to 75 heartbeats (AIx@75) and using a linear regression based on the normalized augmentation index AIx,

In a preferred configuration, the method further comprises the determination of Crest Time (CT), Stiffness Index (SI) and Pulse Area (PA) of the PPG signal and wherein the cardiovascular parameters are estimated with the following equations:
a) vascular age index AgIx:

AgIx=d₀+d₁ [symbol] +d₂p_{age}+d₃p_{height}+d₄med [symbol] HR),

wherein [symbol] is estimated based on characteristic points a, b, c, d, and e:

[symbol]

$$= 45.4 * \frac{b - c - d - e}{a} + 65.9 ;$$

b) pulse wave velocity PWV:

PWV=g₀+g₁ [symbol] +g₂p_{age}+g₃p_{height}+g₄m [symbol] (HR);
c) blood pressure BPdia and BPsys:

BP_{dia}=l_{0d}+l_{1d} [symbol] +l_{2d}m [symbol] (HR)+l_{3d}CTₚ+l_{4d}SIₚ+l_{5d}PAₚ

BP_{sys}=k₀ₛ+k₁ₛ [symbol] +k₂ₛm [symbol] (HR);
d) normalized augmentation index AIx@75:

[symbol]

=(x−y)/y by the sum of two exponential, and

AIx@75=b₀+b₁A [symbol] 5,

wherein AIx@75 is the augmentation index (AIx) normalized to 75 heartbeats; [symbol]
wherein, p_{age} is the age and p_{height} is the body height of the subject, median (HR) is the median heart rate, PTT is the time difference between the PPG pulses, A_{sys} and A_{dia} are magnitudes of the systolic and diastolic peak, respectively, CT is the Crest Time, ST is the Stiffness Index and PA is the Pulse Area of the PPG signal, d₀ to d₄, g₀ to g₄, l_{0d} to l_{kd}, k₀ₛ to k₂ₛ, and b₀ to b₁ represent the coefficients of the respective linear regression equation.

In a preferred configuration, the cardiovascular parameters are estimated based on at least 60 PPG pulses, preferably at least 100 PPG pulses, more preferably at least 120 PPG pulses. The estimation of 60 pulses corresponds to measurement time of approximately 1 minute (with 60 pulses per minute). Therefore, the preferred configurations refer to a measurement time of at least 1 minute (60 PPG pulses), preferably at least 1.7 minutes (100 PPG pulses), more preferably at least 2 minutes (120 PPG pulses). By combining the results obtained by every PPG pulse mediated in the measured time, this allows a more reliable estimation. In this way, if there is a corrupted PPG pulse, its effect can be smoothed if the signals are mediated over the measured time. The measurement of PPG pulses over a defined time has the advantage that the single PPG pulses d₀ not need to be classified as it necessary in the state of the art (e.g. such as in US2013/324859A1) and this provides a more efficient algorithm.

The method according to the present invention allows the estimation of blood pressure and arterial stiffness based on PPG signals. With this invention, new methods to find the characteristic points (features) that are necessary for the estimation in the PPG signal and its time derivatives are proposed. To date no algorithm to achieve this has been available. To find the characteristic points, a model for the PPG waveform is also proposed. After extraction of the features, new models which relate the extracted features to the physiological parameters of interest are provided. Unlike existing methods in the literature, the proposed models according to the present invention allow to incorporate parameters such as height, age and other estimated parameters, such as the heart-rate. In summary, based on advanced algorithms including specific anatomical data, the evaluation of several cardiovascular parameters is achieved. The evaluation of supplementary parameters, such as blood flow, blood pressure, arterial stiffness, vessel elasticity, vascular age allows a comprehensive general health assessment. This individual cardiovascular health assessment reduces the risk of misinterpretation and leads to a more precise health assessment. The measurement of new parameters using PPG sensor technology allows new health production with mobile devices, such as fitness trackers or smartwatches.

It is crucial for the present invention to use two or more PPG sensors at two different positions at the subject, for determining of the cardiovascular parameters pulse wave velocity and blood pressure. The introduction of a second PPG sensor in comparison to the methods described in the prior art has the advantage that the pulse transit time (PTT) can be measured (instead of being estimated), which improves the estimates for the cardiovascular parameters. The use of at least two PPG sensors allows more reliable measurements of cardiovascular parameters.

In one alternative embodiment, one PPG sensor is located at the wrist of the subject and another PPG sensor is located at the fingertip of the subject (which can be included in a mobile device, such as a mobile phone). In another alternative embodiment, one PPG sensor is located at the wrist of the subject and another PPG sensor is located at the wrist of the subject, with a defined distance to the first PPG sensor. It is particularly preferred, when two PPG sensors are located at the wrist of the subject, with a distance of 5 cm or less between the two PPG sensors, preferably 4 cm or less between the two PPG sensors. This allows to include both PPG sensors within one device, which can be worn at the wrist of the subject.

### Assessment of Algorithm for Determination of Cardiovascular Parameters

### Preprocessing of the PPG Signal

The preprocessing phase is an important issue for the correct parameter estimation from the PPG signal. It allows enhancing the PPG wave contour in order to obtain an easier detection of its pivotal points.

Therefore, in an advantageous configuration of the present invention the raw PPG signal from the PPG sensor is processed by one or more of the following:
Normalizing the signal,
Moving average filter, in order to remove the drift, always present in the PPG signal due to breathing,
IV order Chebyshev low-pass filter with zero-phase and cutoff frequency=20 Hz.
   Separation of PPG Signal into Pulses

In order to analyse each individual PPG waveform in the PPG signal and to reduce the effect of motion artefacts, the PPG signal is not examined as a whole but in sections. According to the present invention the signal is divided into individual pulses, as all features which are extracted from the PPG signal can be derived from one pulse wave. The systolic foot is the most prominent feature of a PPG pulse and can therefore be found most reliably in the PPG signal. Therefore, the PPG signal was chopped into PPG pulses at this systolic foot by finding the minima in the PPG signal. This strategy allows to analyse each pulse individually. If a few pulses are not correctly recognized, this does not have a falsifying effect on the final results for a measurement as the final parameter values are calculated by the median of all individual pulses' results.

To determine the different cardiovascular parameters, the PPG waveform needs to be analysed and different features are extracted from the PPG waveform.

### Parameter Estimates

### 1. Augmentation Index (AIx_{PPG}):

An indirect measure of arterial stiffness can be provided by the Augmentation Index (AIx). It provides information about the pressure wave reflection by the peripheral circulatory system. The Augmentation Index measure was transposed from the Blood Pressure Pulse Wave Analysis to the PPG signal, assuming that one is able to obtain information about the arterial stiffness analyzing the PPG waveform.

The PPG pulse wave is not a pressure pulse wave. Thus, the augmentation index as described above be obtained directly from the PPG signal. Generally, the Augmentation Index can be estimated thanks to the PPG morphological properties. According to literature, the augmentation index is calculated with the help of the following formula:

$$\begin{matrix}AIx = \frac{y}{x} & ( 1.1 ) \\ AIx = \frac{x - y}{x} & ( 1.2 )\end{matrix}$$

wherein y is the diastolic peak amplitude and x is the systolic peak amplitude (as shown in FIG. 1.2).

The AIx describes the augmentation of the PPG signal from the systolic to the diastolic peak.

From the PPG pulse wave, the systolic A_{sys} and diastolic A_{dia} peak amplitudes are estimated (corresponding to x and y in formula 1.2 respectively), as well as their times tₛ and t_{d}. The determination of A_{dia} in the PPG waveform can be very difficult when the reflected wave is very small and there is no visible diastolic peak in the waveform (see FIG. 1.2). To still be able to estimate both peak positions, two different methods to model the form of the two waves were developed.

In the first method, the PPG waveform is modelled as a sum of the two pulse waves through exponential functions.

$$\begin{matrix}{y}_{pulse} ( t ) = {y}_{sys} ( t ) + {y}_{dia} ( t ) \\ = {b}_{1} {e}^{\frac{- {( t - {\hat{t}}_{s} )}^{2}}{{b}_{2}}} + {b}_{3} {e}^{\frac{- {( t - {\hat{t}}_{d} )}^{2}}{{b}_{4}}}\end{matrix} \qquad ( 1.3 )$$

Nonlinear regression is applied to fit the model to the PPG waveform and receive estimates of tₛ and t_{d} to find A_{sys} and A_{dia}, respectively.

The second method makes use of the fact that the maximum in the PPG waveform is the systolic peak. By modelling only the first wave with known position at the systolic peak, its exponential model is subtracted from the PPG signal and yield the remaining reflected wave,

$$\begin{matrix}{y}_{d i a} ( t ) = {y}_{pulse} ( t ) + {y}_{s y s} ( t ) \\ = {y}_{pulse} ( t ) {b}_{1} {e}^{\frac{- {( t - {\hat{t}}_{s} )}^{2}}{{b}_{2}}}\end{matrix} \qquad ( 1.4 )$$

whose maximal value max y_{dia}(t)=A_{dia} and t_{d} is the corresponding diastolic time index estimate (see FIG. 1.5).

A parameter that seems to be more reliable is the Augmentation Index normalized to 75 heartbeats (AIx@75). Indeed, it seems that this parameter depends on the heartbeat. It was introduced for the first time in the work of Wilkinson et al. (American Journal of Hypertension, vol. 15, pp. 24-30, 2002). It has been found that the AIx estimated from the Blood Pressure wave has different values compared to the same parameter estimated from the PPG wave. Thus, the AIx and the AIx@75 were used in a linear regression with the reference values. Same methods were applied to calculate both the AIx and AIx@75.

The normalized index value AIx@75 was obtained and in used in linear regression model:

AIx@75=b₀+b₁A [symbol] 5; (1.5)

Feature Extraction from Signal's Derivatives

Other features are obtained from the signal's derivatives which are calculated by the differences between adjacent samples. A moving average filter was applied to remove high frequency noise introduced by taking the derivative. To reliably find the characteristic points a to e, an algorithm to find the two most prominent maxima was developed and they were marked as a and e, respectively. The point c is then the most prominent peak between point a and e. Furthermore, point b is the most prominent minimum in the second derivative and point d is the most prominent minimum between points c and e (see FIG. 1.6).

Therefore, in a preferred embodiment of the present invention the characteristic points a, b, c, d, and e are automatically derived from the second derivative of the PPG pulse, wherein a and e are the first and second most prominent maxima in the second derivative, respectively, c is the most prominent peak between the points a and e, b is the most prominent minimum in the second derivative and, d is the most prominent minimum between points c and e.

### 2. Vascular Age Index (AgIx_{PPG}):

Regarding the PPG waveform, a Vascular Age Index estimate can be obtained through the analysis of the second derivative of the PPG signal, also known as Acceleration Photoplethysmography (APG). It is characterized by several landmark points, like the PPG wave; the estimation of these points is used to obtain indicators that give information about the cardiovascular function, including the Vascular Age Index. The state-of-the-art literature calculates a ratio of the characteristic points by

$$= 45.5 * \frac{b - c - d - e}{a} + 65.9 \qquad ( 1.6 )$$

The index describes the cardiovascular age of a person. It should be lower than the person's chronological age if their vessels aged slower than average and higher than their chronological age otherwise.

Despite the most used parameter from the APG is the Vascular Age Index, other measures have been investigated starting from the APG wave estimates, for example, ratios between the b, c, d or e wave and a wave in several studies (Elgendi, Current Cardiology Reviews, vol. 8, pp. 14-25, 2012). It has been found that these ratios vary with the subject age. As a Vascular Age Index alternative, in case of the c and d waves are not visible, the (b-e)/a ratio could be used, as suggested in another study (Baek et al., 6th International Special Topic Conference on Information Technology Applications in Biomedicine, 2007).

In addition to the Vascular Age Index, this index was also estimated:

$$\frac{b - e}{a} \qquad ( 1.7 )$$

To more reliable estimate AgIx, a new linear regression model with coefficients dᵢ based on the estimated Vascular Age Index [symbol] , which is based on the characteristic points a, b, c, d and e was developed:

AgIx=d₀+d₁ [symbol] +d₂p_{age}+d₃p_{height}+d₄me [symbol] HR) (1.8)

wherein dᵢ are the coefficients, p_{age} is the age, p_{height} is the height, me [symbol] HR) is the median heart rate estimate of a person.

[symbol]

### Pulse Wave Velocity (PWV):

The PWV is measured experimentally as the ratio between the distance between two different measurement sites on the same line through which the pressure wave propagates, and the time interval between wave corresponding points.

The Pulse Wave Velocity can be estimated also with the PPG signal. In this case, the PWV can be obtained with two different instrumental setups:
ECG+PPG sensor: one has to evaluate the Pulse Arrival Time (PAT) as the time interval between the ECG R peak and a PPG landmark point (systolic foot, max gradient or systolic peak);
2 PPG sensors: they are positioned one downstream of the other and, in this case, one has to evaluate the Pulse Transit Time (PTT) as the time interval between the two measurement sites [21].

It is necessary to distinguish and specify the measured time interval: the PAT is equal to the sum of PTT and the Pre-Ejection Period (PEP), that is the time interval between the beginning of the ventricular depolarization and the moment in which the aortic valve opens. Since PEP is difficult to measure or predict and is not a linear function of pressure, it turns out that PAT is a less accurate indicator than the PTT. Although it is more difficult to assess, PTT provides a better measure for monitoring. This parameter would allow estimating the aortic PWV (the aorta is the reference point to measure the PWV in the literature). Modern pressure measurement systems also calculate aortic PWV with indirect methods.

To obtain a PWV estimate, PPG signals systolic feet from two different measurement systems are identified. Thanks to the difference between the time instants at which the systolic feet are recorded, it is possible to know the Pulse Arrival Time and the Pulse Transit Time, depending on the instruments (ECG and PPG in the first case, two PPG signals in the second). This measure will be used to evaluate the correlation between the PAT or the PTT and the Pulse Wave Velocity measured from the gold standard instrument, which refers to the central PWV, i.e. in the aorta. For this reason, a linear regression was created using Pulse Transit Time values, age, height, median heart rate value and three typical parameters of the PPG signal, i.e. Crest Time, Stiffness Index and Pulse Area.

The PWV is estimated by the time difference between pulses of two PPG signals measured at two separately placed PPG sensors (here the PTT). Therefore, the time difference between the systolic feet of the signals is examined. The median time differences are used for a linear regression model to estimate the PWV. Additional physiological and personal data were further included in the linear regression model:

PWV=g₀+g₁ [symbol] +g₂p_{age}+g₃p_{height}+g₄me [symbol] (HR) (1.9)

wherein gᵢ are the coefficients, PTT is the time difference between the PPG pulses, p_{age} is the age, p_{height} is the height and me [symbol] HR) is the median heart rate of a person.

It is preferred that two PPG signals are measured and the time difference between the two corresponding PPG pulses are considered. In one embodiment, one PPG sensor can be positioned at the wrist of a user and the second sensor can be positioned at the finger of a user. However, in an advantageous configuration, two PPG sensors can be positioned at the wrist of a user with a certain distance between both sensors. This allows the implementation in wrist-worn devices, such as smartwatches or fitness trackers.

### Blood Pressure (Bp):

The blood pressure estimate from the PPG signal is not such a trivial task. Previous studies suggest to estimate the BP by a simple linear regression model using the extracted systolic and diastolic times of a PPG pulse:

BP_{dia}=a_{SBP}t_{dia}+b_{SBP} (1.10)

BP_{sys}=a_{DBP}t_{sys}+b_{DBP} (1.11)
Wherein a_{SBP}, b_{SBP}, a_{DBP} and b_{DBP} are coefficients that have to be estimated based on reference values.

For the present invention a strategy for estimating the arterial blood pressure (systolic and diastolic) was developed, working on the Pulse Transit Time and evaluating the linear regression of these values with the blood pressure estimates obtained with the gold standard instrument. Furthermore, other parameters were used in the linear regression estimates, like the median heart rate, Crest Time, Stiffness Index and Pulse Area and physiological parameters, such as age and height.

BP_{sys}=k₀ₛ+k₁ₛ [symbol] +k₂ₛp_{age}+k₃ₛp_{height}+k₄ₛm [symbol] (HR) (1.12)

BP_{dia}=k_{0d}+k_{1d} [symbol] +k_{2d}p_{age}+k_{3d}p_{height}+k_{4d}m [symbol] (HR) (1.13)

BP_{sys}=l₀ₛ+l₁ₛ [symbol] +l₂ₛm [symbol] (HR)+l₃ₛCTₚ+l₄ₛSIₚ+l₅ₛPAₚ (1.14)

BP_{dia}=l_{0d}+l_{1d} [symbol] +l_{2d}m [symbol] ((HR)+l_{3d}CTₚ+l_{4d}SIₚ+l_{5d}PAₚ (1.15)
wherein k₀ₛ to k₂ₛ, k_{0d} to k_{2d}, l_{0d} to l_{5d}, l₀ₛ to l₅ₛ, are the coefficients, [symbol] is the time difference between the PPG pulses, p_{age} is the age, p_{height} is the height and me [symbol] HR) is the median heart rate of a person, CTₚ is the Crest Time, SIₚ is Stiffness Index and PAₚ is the Pulse Area of the PPG signal from the proximal sensor.

### Heart Rate Variability (HRV):

The heart rate variability (HRV) describes the variation in the time interval between heartbeats. The interbeat interval (IBI) value for each heartbeat is estimated as the time interval between two corresponding landmark points of two consecutive PPG waves (systolic foot, max gradient or systolic peak). In FIG. 1.7, for instance, the IBI is measured as the time interval between two consecutive systolic feet.

Once the IBIs have been measured, it is possible to estimate the HRV parameters. Conventionally, HRV analysis is performed in the time domain and in the frequency domain. In addition, some of these parameters can only be estimated if the recording has a sufficiently long duration. For short recordings (i.e. two minutes at least), the following are some of the possible indices that can be obtained (Shaffer and Ginsberg, Frontiers in Public Health, vol. 5, n. 258, p. 17 pp, 2017):
1. Standard Deviation of the IBI of normal sinus beats (SDNN)
2. Number of adjacent intervals that differ from each other by more than 50 ms (NN50 and pNN50)
3. Root Mean Square of Successive Difference between normal heartbeats (RMSSD), obtained by first calculating each successive time difference between heartbeats; then, each of the values is squared and the result is averaged before the square root of the total
4. LF/HF ratio, the ratio between the low-frequency power (0.04-0.15 Hz) and the high-frequency power (0.15-0.4 Hz)
5. Poincaré Plot, it is obtained by plotting every IBI interval against the prior interval, creating a scatter plot; the Poincaré Plot can also be analyzed by fitting an ellipse to the plotted points. After the fitting phase, two non-linear measurements can be obtained:
   5.a. SD1: standard deviation of the distance of each point from the x-axis, specifies the ellipse's width; it reflects short-term HRV
   5.b. SD2: standard deviation of each point from the y=x+mean(IBI interval), it specifies the ellipse's length; it measures the short- and long-term HRV
6. Sample Entropy, which measures the regularity and complexity of the time series.

An increasing number of wearable devices claim to provide accurate, economic and easily measurable HRV indices using PPG technique. Several studies have focused on the reliability of the HRV indices reported by PPG measurements compared to the gold standard, given by the ECG signal. In particular, in a recent review (Georgiou et al., Folia Medica, vol. 60, n. 1, pp. 7-20, 2018) the result that emerges is that PPG technology can be a valid alternative for HRV measurements, although it is still necessary to conduct more in-depth studies under non-stationary conditions.

According to the present invention one or more cardiovascular parameters are calculated by measuring two or more PPG signals with two or more PPG sensors and using advanced algorithms to determine vascular age index AgIx, blood pressure BPdia and BPsys, pulse wave velocity PWV and augmentation index AIx.

In one configuration, only one cardiovascular parameter is measured, either the Augmentation index AIx is determined or only the Vascular age index AgIx is, or only Blood pressure is determined or only Pulse wave velocity PWV is determined.

In further configurations, two cardiovascular parameters are measured, either Augmentation index AIx and the Vascular age index AgIx are determined. In further alternatives, additionally the Blood pressure is determined or Pulse wave velocity PWV or both are determined.

In further configurations Augmentation index AIx and Blood pressure are determined. In further alternatives, additionally the Vascular age index AgIx is determined or Pulse wave velocity PWV or both are determined.

In further configurations Augmentation index AIx and Pulse wave velocity PWV are determined. In further alternatives, additionally the Vascular age index AgIx is determined or Blood pressure or both are determined.

In further configurations Vascular age index AgIx and Blood pressure are determined. In further alternatives, additionally the Vascular age index AgIx is determined or Augmentation index AIx or both are determined.

In further configurations Vascular age index AgIx and Pulse wave velocity PWV are determined. In further alternatives, additionally Blood pressure is determined or Augmentation index Aix or both are determined.

In further configurations Blood pressure and Pulse wave velocity PWV are determined. In further alternatives, additionally Augmentation index Aix is determined or Vascular age index AgIx or both are determined.

In a preferred configuration, the cardiovascular parameters Augmentation index AIx, Vascular age index AgIx, Blood pressure and Pulse wave velocity PWV are determined.

In a particularly preferred configuration, the cardiovascular parameters Augmentation index AIx, Vascular age index AgIx, Blood pressure and Pulse wave velocity PWV are determined.

In alternative configurations, additionally to one, two, three or four cardiovascular parameters, the heart rate variability HRV is determined by calculating one or more of the following
Minimum and maximum interbeat interval (IBI)
Median and mean IBI
Minimum and maximum heart rate
Median and mean heart rate
Standard Deviation of the IBI of normal sinus beats (SDNN)
Number of adjacent intervals that differ from each other by more than 50 ms (NN50 and pNN50)
Root Mean Square of Successive Difference between normal heartbeats (RMSSD),
LF/HF ratio, the ratio between the low-frequency power (0.04-0.15 Hz) and the high-frequency power (0.15-0.4 Hz)
SD1: standard deviation of the distance of each point from the x-axis in a Poincaré Plot, obtained by plotting every IBI interval against the prior interval
SD2: standard deviation of each point from the y=x+mean (IBI interval) in a Poincaré Plot, obtained by plotting every IBI interval against the prior interval
Sample Entropy.

The present invention can be applied using PGG sensors which are included in a number of different human body health monitoring devices, such as wrist-type fitness trackers, smartwatches or special devices used by medical professionals. The method according to the present invention allows the detailed analysis of the cardiovascular condition of a person with the help of simple wrist-worn devices by analyzing several cardiovascular parameters.

Therefore, in an advantageous configuration of the present invention one or more calculated parameters are displayed on a human body health monitoring device, which includes at least one PPG sensor.

In an alternative configuration, one or more calculated parameters are displayed on a human body health monitoring device, which includes at least two PPG sensors, thereby allowing the evaluation of one or more cardiovascular parameters by analysing the time difference between two PPG signals.

In another preferred embodiment of the present invention, an acoustic or visual signal is outputted together with the calculated parameter.

In another alternative embodiment of the present invention, one or more calculated parameters are displayed on a human body health monitoring device, which contains at least two PPG sensors.

In an alternative embodiment of the present invention the calculated cardiovascular parameters are compared with prestored cardiovascular index parameters and an acoustic or visual signal is outputted, if the calculated cardiovascular parameters differ more than X % from the prestored cardiovascular index parameters, whereas X is chosen from the following values: 5, 10, 20, 30, 40, 50, 60, 70, 80, 90, 100.

Another aspect of the present invention is related to a wrist-worn device for determining one or more of the following parameters:
the vascular age index AgIx,
the pulse wave velocity PWV,
blood pressure BPdia and BPsys,
augmentation index AIx,
   wherein the device comprises
two PPG sensors, with a distance of 5 cm or less, facing the dorsal part of the arm,
wherein the PPG sensor comprises at least one green light source and comprises a sampling frequency of preferably 512 Hz.

In a preferred embodiment, the device further comprises signal processing means adapted to calculate one or more of the following:
the vascular age index AgIx using linear regression based on the characteristic points a, b, c, d, and e, age (page), body height (p_{height}) and median heart rate of the subject,
the pulse wave velocity PWV using linear regression based on the time difference between the two PPG pulses (PTT), age (page), body height (p_{height}) and median heart rate estimation of the subject,
blood pressure BPdia and BPsys using linear regression based on time difference between the two PPG pulses (PTT) and median heart rate and
optionally the augmentation index AIx, based on the systolic Asys and diastolic Adia peak amplitudes normalized to 75 heartbeats (AIx@75) and using a linear regression based on the normalized augmentation index AIx,

The wrist-worn device can be a fitness tracker or a smartwatch.

### EXAMPLES

### Experimental Setup

Two different instrumental setups were used for the experiments for the present invention.

### GTEC Setup

A polygraph setup comprising the g.MOBllab, a portable biosignal acquisition and analysis system from G.TEC medical engineering GmbH, Austria was used («gMOBIIab Instructions For Use» [Online]. Available: http://www.gtec.at/Download/Product-Manuals-Handbooks/g.MOBIIab/gMOBIIabInstructionsForUse.)

In the GTEC setup, two PPG sensors are connected to the g.MOBIIab via a Connector Box. The electrodes for the ECG are, instead, directly connected to it. The g.MOBIIab can communicate with the software that records and displays signals through a Bluetooth connection. Once the signals are recorded, they are stored in a folder specified by the user.

The relevant information that is acquired with this instrumental setup is:
ECG signal [mV]
PPG signal from finger sensor [a.u.]
PPG signal from lobe sensor [a.u.]
Initial time of recording in Unix Time1 [ms]

Other relevant information is the following:
Sampling frequency: 256 Hz (non-customizable)
PPG sensors
   IR light source
   1 photodetector

Thanks to this instrumental setup, the signals from the two PPG sensors are recorded simultaneously via the main station (g.MOBllab), along with the electrocardiogram signal. This allows a perfect synchronization, both between the two PPG sensors and among the PPG sensors and the ECG signal. This feature would allow, therefore, to accurately derive the cardiovascular parameters related to the temporal properties of the PPG signal. However, two problems arise:
i. The two sensors are equipped with an infrared light source. As reported in the previous chapter, a light source like this is not the most suitable for acquisitions on the wrist, so the recorded signal has a poor quality; despite this, it is still possible to identify the systolic feet
ii. As reported previously (O'Rourke et al., American Journal of Hypertension, vol. 15, pp. 426-444, 2002), Pulse Wave Velocity between the brachial and radial artery ranges from about 800 cm/s to 1160 cm/s. Anyway, it is necessary to consider that these reference values are for 40-year-old subjects, so the lower limit may be smaller than 800 cm/s. If the Pulse Wave Velocity is higher than 1050 cm/s, the GTEC system would not be able to detect it, as the sampling frequency is equal to 256 Hz and, therefore, the sampling period is 0.0039 s. Indeed, if the sensors are placed 4 cm apart from each other, the maximum velocity that can be detected is 1050 cm/s because, in this way, the pulse takes 0.0039 s to travel 4 cm. In order to record a signal with a velocity higher than 1050 cm/s, a lower sampling period would be necessary.

### E4 Setup

The Empatica E4 wristband (Empatica Inc., United States) was used («E4 wristband user's manual» [Online]. Available: https://empatica.app.box.com/v/E4-User-Manual).

The streaming mode was chosen as acquiring signal mode. In the streaming mode, once the E4 wristband is worn on the wrist, a Bluetooth connection is established via a “Bluegiga Bluetooth Smart Dongle” module, the only connection type supported by the E4. The data are then sent to the E4 streaming server, which, in turn, sends the data streaming through a TCP connection. Data are then saved in a specific folder. The relevant information that is acquired with this instrumental setup is PPG signal [a.u.] and recording instant for each value in Unix Time [ms].

Additional relevant information is the following:
Sampling frequency: 64 Hz (non-customizable)
PPG sensor
   4 light sources, 2 green and 2 red
   2 photodetectors
   The internal algorithm to remove motion artefacts.

In the streaming mode, technological limits have been detected, which also d₀ not allow a perfect synchronization between the two sensors. The delay introduced by the multiple connections (i.e. Bluetooth and TCP) is not deterministic, and, therefore, cannot be treated as an offset to be deleted once the signal analysis phase has been reached. The advantage introduced by the E4 wristband, on the other hand, results in a high signal quality, which can be reached thanks to several factors:
i. Four light sources and two photodetectors: the E4 system combines the signals acquired by the two photodetectors at different wavelengths (green and red), obtaining a signal that is less sensitive to ambient lighting
ii. Internal algorithm: each E4 wristband has an internal algorithm, owned by Empatica, which performs an initial preprocessing on the signal. On one side, this could represent an advantageous feature of this technology but, on the contrary, it could be a further cause of delays that are difficult to quantify

The two systems (GTEC and E4) were combined for the present study. This is possible because three of the parameters to be obtained (i.e. Augmentation Index, Vascular Age Index, and Heart Rate Variability) depend on the morphological characteristics of the PPG wave, and the remaining two measures (i.e. Pulse Wave Velocity and Blood Pressure) depend on the temporal distance between two PPG waves from two different sensors. Therefore, it was decided to use both systems at the same time, exploiting the best properties from each of them: the high quality of the E4 wristband, and the synchronization from the GTEC system.

### In Vivo Study

An experimental study has been conducted, with the instrumental setup discussed in the previous chapter, to perform an assessment of the cardiovascular parameters estimates using the PPG signals. The evaluation was conducted on 20 healthy subjects, whose data on age, height, and weight are reported in Table 1.

TABLE 1
Descriptive statistics of the analyzed population

| | Mean | St.Dev. |
|---|---|---|
| Age | 36.2 | 11.1 |
| Height [cm] | 170.2 | 11.7 |
| Weight [kg] | 64.75 | 12.5 |

The experimental setup is the following:
Two Empatica E4 wristbands
GTEC polygraph, equipped with:
g.MOBllab
2 PPG sensors with IR light source
ECG cables

In order to validate the measurements obtained with this instrumental setup, a gold standard instrument was used, the Mobil-O-Graph PWA 24 h which is a clinical device by I.E.M. GmbH («Mobil-O-Graph 24 h PWA user's manual» [Online]. Available: www.iem.de/_attic/website/UserManual_NG_HMS-CS_24 h-PWA_EN.pdf). This device works similar to a standard measurement device for blood pressure, applying a cuff to the subject's upper arm. It allows performing the Pulse Wave Analysis on the pressure wave, obtaining the correct values for each index.

The experimental protocol has been applied to 20 subjects. It consists of the following steps:
1. 5 minutes of rest
2. Sensors placement
   a. 2 E4 wristband 4 cm apart from each other on the dorsal part of the wrist
   b. 2 GTEC PPG sensors between the E4 strap and the ventral part of the arm, 4 cm apart from each other
3. E4 wristbands Bluetooth connection
4. GTEC system Bluetooth connection
5. 2:30 minutes acquisitions
6. Sensors removal
7. Placement of the Mobil-O-Graph blood pressure cuff
8. Mobil-O-Graph Bluetooth connection
9. Blood pressure signal acquisition with “Triple Pulse Wave Analysis” mode

### Preprocessing of the PPG Signal

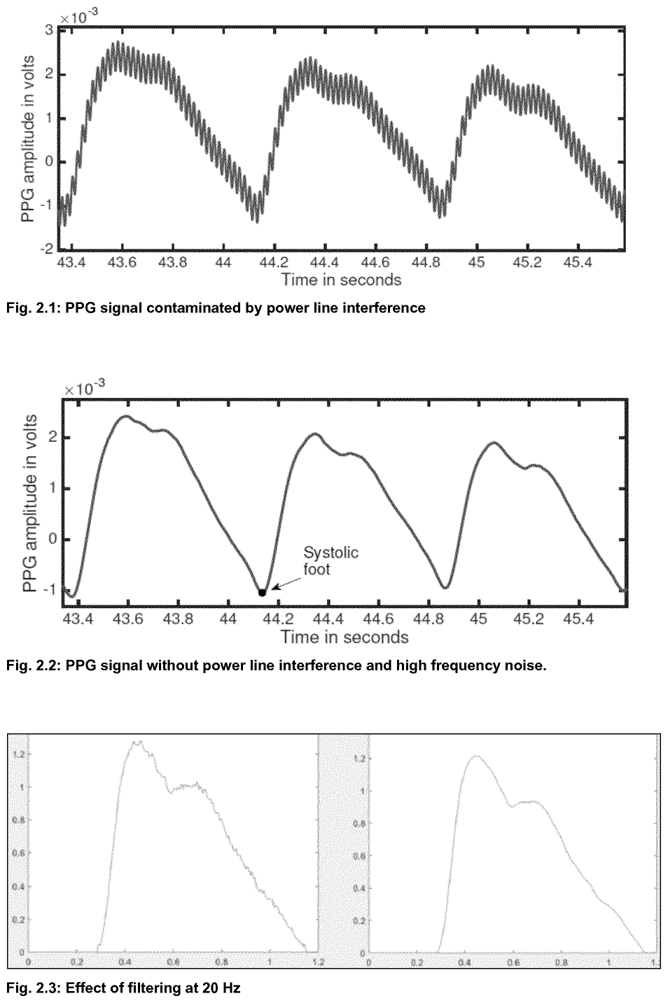

The preprocessing phase is an important issue for the correct parameter estimation from the PPG signal. It allows enhancing the PPG wave contour in order to obtain an easier detection of its pivotal points. A PPG signal that is measured without any modification usually contains a visible power line interference at a frequency of 50 Hz, as displayed in FIG. 2.1. A notch filter at 50 Hz is used to remove this interference. A PPG signal cleaned from power line interference and high frequency noise is displayed in FIG. 2.2.

A combined preprocessing algorithm was chosen [34] [35], which consists of three steps:
i. Signal normalization:

$$P P {G}_{n o r m} = \frac{P P {G}_{r a w} - mean ( {PPG}_{r a w} )}{s t d e v ( P P {G}_{r a w} )} \qquad ( 1.16 )$$

ii. Moving average filter, in order to remove the drift, always present in the PPG signal due to breathing
iii. IV order Chebyshev low-pass filter with zero-phase and cutoff frequency=20 Hz

The step filtering at 20 Hz, the filter allows to remove the irregularities of the signal, which d₀ not provide additional information; these fast fluctuations risk to be detrimental to the estimation of the correct position of the systolic peaks and the diastolic peaks (shown in FIG. 2.3).

### Evaluation Metrics

The accuracy and reliability of the proposed algorithms was validated by comparing the estimates of those algorithms with measurements of a reference device that is clinically approved. These measurements, obtained by the Mobil-O-Graph from I.E.M. GmbH, serve as reference values and can themselves differ from the true value, as the device also has an intrinsic measurement error and thus fluctuates in its measurement values. To reduce the influence of the intrinsic measurement error of the reference device, three consecutive measurements were taken with the reference device and the median of those three values for each cardiovascular parameter were calculated.

The signals were processed in Matlab. The algorithm for performance assessment involves three major steps:
1. Parameters estimation from PPG signals
2. Linear regression analysis of the estimated parameters
3. Assessment of the agreement between the estimated parameters and gold standard measures (table below)

The performances of the parameter estimates from the PPG signals were evaluated based on five indicators. For validation, five different metrics were calculated, the mean error, the standard deviation (STD), the mean absolute error (MAE), the mean squared error (MSE) and the root-mean-squared error (RMSE), where yₑₛₜ(i) is the estimated cardiovascular parameter of interest with length N=242 equal to the total number of measurements for all participants and y_{ref}(i) is the reference value of the cardiovascular parameter thereof:

$$mean error \text{:} MEAN = \frac{1}{N} {\sum }_{i}^{N} ( {y}_{e s t} ( i ) - {y}_{r e f} ( i ) )$$

$$standard deviation ( STD ) \text{:}$$

$$STD = \sqrt{\frac{1}{N - 1} {\sum }_{i}^{N} {( {y}_{e s t} ( i ) - {y}_{r e f} ( i ) )}^{2}}$$

$$mean absolute error ( MAE ) \text{:} MAE = \frac{1}{N} {\sum }_{i}^{N} \vert {y}_{e s t} ( i ) - {y}_{r e f} ( i ) \vert$$

$$mean squared error ( MSE ) \text{:} MSE = \frac{1}{N} {\sum }_{i}^{N} {( {y}_{e s t} ( i ) - {y}_{r e f} ( i ) )}^{2} \\ root \text{-} mean \text{-} squared error ( RMSE ) \text{:} \\ RMSE = \sqrt{M S E} = \sqrt{\frac{1}{N} {\sum }_{i}^{N} {( {y}_{e s t} ( i ) - {y}_{r e f} ( i ) )}^{2}}$$

Once the method and the best sensor position are decided, the nonparametric Spearman's rank correlation coefficient ρ is then evaluated, with its p-value.

Estimation of linear regression coefficients to estimate cardiovascular parameters

### 1. Augmentation Index Aix

Augmentation Index estimates were obtained using the methods described before:
Method 1.1: AIx=y/x by the sum of two exponential
Method 1.2: AIx=(x−y)/x by the sum of two exponential
Method 2.1: AIx=y/x by modeling a single exponential
Method 2.2: AIx=(x−y)/x by modeling a single exponential

Although the mean error is low (basically, it is zero), the standard deviation is relatively high so that this kind of measure could not be defined as a reliable parameter. For this reason, it was decided to assess the performance of the normalized index, the AIx@75. Tables 2.1 and 2.2 show the results obtained from the PPG signal recorded, respectively, by the proximal and the distal sensors.

Table 2.1
Results for Augmentation Index @ 75 from proximal E4 sensor

| Proximal E4 | MEAN (%) | STD (%) | MAE (%) | MSE (%²) | RMSE (%) |
|---|---|---|---|---|---|
| Method 1.1 | −6.70E−12 | 9.9482 | 7.9047 | 94.0184 | 9.6963 |
| Method 1.2 | −6.25E−11 | 8.7559 | 7.2471 | 72.832 | 8.5342 |
| Method 2.1 | −6.70E−12 | 9.9482 | 7.9047 | 94.0184 | 9.6963 |
| Method 2.2 | −1.01E−09 | 9.006 | 7.0456 | 77.0528 | 8.778 |

TABLE 2.2
Results for Augmentation Index @ 75 from proximal E4 sensor

| Distal E4 | MEAN (%) | STD (%) | MAE (%) | MSE (%²) | RMSE (%) |
|---|---|---|---|---|---|
| Method 1.1 | −6.05E−12 | 10.0644 | 7.8544 | 96.2268 | 9.8095 |
| Method 1.2 | −1.49E−11 | 8.5497 | 7.1943 | 69.4419 | 8.3332 |
| Method 2.1 | −6.05E−12 | 10.0644 | 7.8544 | 96.2268 | 9.8095 |
| Method 2.2 | −1.36E−09 | 9.8811 | 7.9795 | 92.7551 | 9.6309 |

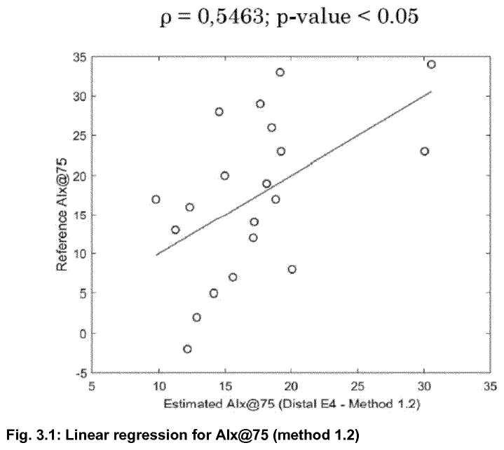

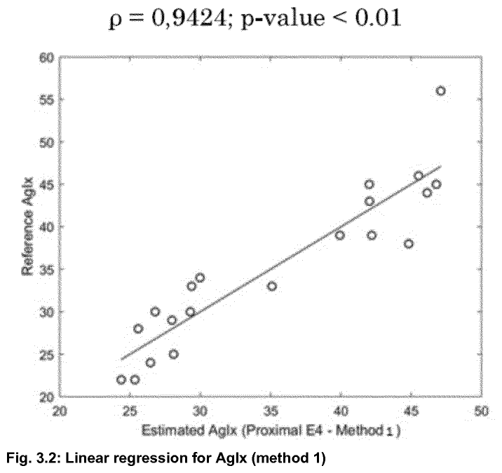

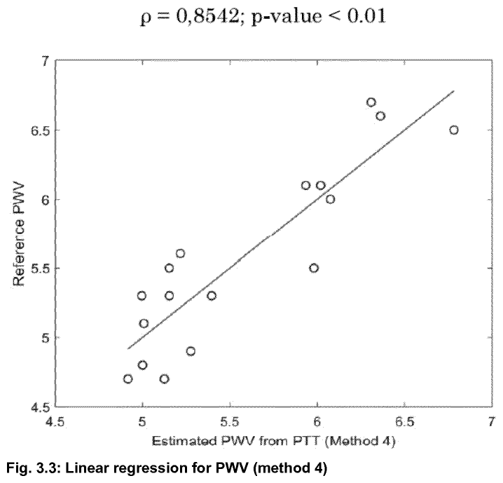

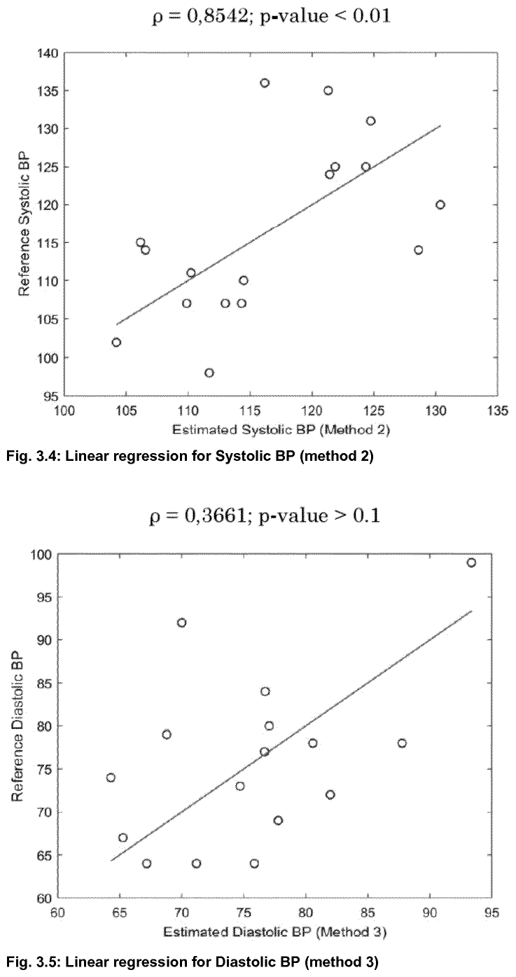

The indicators are all significantly lower than those obtained for AIx. The best results are achieved thanks to the Method 1.2 using the PPG signal from the distal sensor. FIG. 3.1 shows the values obtained by this method in relation to the reference values, the Spearman correlation coefficient and its p-value.

### 2. Vascular Age Index AqIx_{PPG}

Three different methods for estimating the Vascular Age Index have been evaluated:
Method 1: the APG amplitude waves are obtained finding maximum and minimum peaks. Then the formula (1.6) is used to estimate the Vascular Age Index;
Method 2: an additional method found in the literature [16] [36] has been implemented. Formula (1.6) was used also in this case;
Method 3: the APG landmark points were detected as in Method 2, but the Vascular Age Index from the gold standard instrument was compared with the amplitude ratios obtained through formula (1.7).

Tables 3.1 and 3.2 show the values obtained for the proximal and distal sensors. The vascular age index is given in years (y) and so are its estimation errors.

TABLE 3.1
Results for Vascular Age Index from proximal E4 sensor

| | MEAN | STD | MAE | MSE | RMSE |
|---|---|---|---|---|---|
| Proximal E4 | (y) | (y) | (y) | (y²) | (y) |
| Method 1 | −4.11E−12 | 9.1741 | 7.4694 | 79.9558 | 8.9418 |
| Method 2 | −5.90E−12 | 9.3149 | 7.7572 | 82.4293 | 9.0791 |
| Method 3 | −1.27E−08 | 9.2439 | 7.6288 | 81.1769 | 9.0098 |

TABLE 3.2
Results for Vascular Age Index from distal E4 sensor

| | MEAN | STD | MAE | MSE | RMSE |
|---|---|---|---|---|---|
| Distal E4 | (y) | (y) | (y) | (y²) | (y) |
| Method 1 | −3.87E−01 | 9.2009 | 7.6123 | 80.5736 | 8.9763 |
| Method 2 | −3.46E−10 | 9.3163 | 7.7577 | 82.4544 | 9.0804 |
| Method 3 | −7.90E−11 | 9.3085 | 7.7408 | 82.3159 | 9.0728 |

There are no particular differences between the two PPG recording sites. The Vascular Age Index estimated from the APG amplitude waves does not seem to provide very accurate results. For this reason, it was decided to introduce, in the linear regression, other regressors, which are: Age, Height and Median heart rate, see formula (1.8). The results are presented in tables 3.3 and 3.4.

TABLE 3.3
Results for Vascular Age Index with metadata from proximal E4 sensor

| | MEAN | STD | MAE | MSE | RMSE |
|---|---|---|---|---|---|
| Proximal E4 | (y) | (y) | (y) | (y²) | (y) |
| Method 1 | −1.18E−11 | 3.5413 | 2.8271 | 11.9137 | 3.4516 |
| Method 2 | −1.48E−11 | 3.8937 | 3.1198 | 14.4031 | 3.7951 |
| Method 3 | 9.91E−11 | 4.7023 | 3.373 | 15.7547 | 3.9692 |

TABLE 3.4
Results for Vascular Age Index with metadata from distal E4 sensor

| | MEAN | STD | MAE | MSE | RMSE |
|---|---|---|---|---|---|
| Distal E4 | (y) | (y) | (y) | (y²) | (y) |
| Method 1 | 8.56E−12 | 4.0471 | 3.1751 | 15.5598 | 3.9446 |
| Method 2 | 1.42E−01 | 4.1293 | 3.4354 | 16.2187 | 4.0272 |
| Method 3 | −9.96E−12 | 4.1438 | 3.4203 | 16.3123 | 4.0388 |

Performances improve considerably by incorporating additional subjects' information in the linear regression estimate. The standard deviation values are acceptable. The best method is Method 1 when used to the signal of the proximal PPG sensor. FIG. 3.2 shows the values obtained by this method compared to reference values, the Spearman correlation coefficient and its p-value are also shown.

### 3. Pulse Wave Velocity PWV

The Pulse Wave Velocity estimates have been obtained only using the GTEC system. The results based on three different set-ups have been evaluated:
Pulse Arrival Time between the ECG R peak and the consecutive PPG systolic foot from the proximal GTEC sensor
Pulse Arrival Time between the ECG R peak and the consecutive PPG systolic foot from the distal GTEC sensor
Pulse Transit Time between the systolic feet of two PPG waves recorded by the proximal and distal GTEC sensor, respectively.

Furthermore, linear regressions have been used. We tested the performances of 6 models:
Model 1: PAT or PTT only
Model 2: PAT or PTT+age
Model 3: PAT or PTT+age+height
Model 4: PAT or PTT+age+height+median(HR), formula (1.9)
Model 5: PTT+Crest Time+Stiffness Index+Pulse Area (from PPG proximal sensor)
Model 6: PTT+Crest Time+Stiffness Index+Pulse Area (from PPG distal sensor)

The results for PWV obtained from PAT and PTT were then compared.

Tables 4.1, 4.2 and 4.3 show the estimated PWV values with the three different instrumental set-ups.

TABLE 4.1
Results for Pulse Wave Velocity from ECG-Proximal GTEC sensor

| ECG-Prox | MEAN | STD | MAE | MSE | RMSE |
|---|---|---|---|---|---|
| GTEC | (m/s) | (m/s) | (m/s) | (m/s²) | (m/s) |
| Model 1 | −8.57690E−10 | 0.7721 | 0.6404 | 0.5663 | 0.7525 |
| Model 2 | −1.30E−13 | 0.3772 | 0.2881 | 0.1351 | 0.3676 |
| Model 3 | −3.43E−12 | 0.3718 | 0.2929 | 0.1313 | 0.3623 |
| Model 4 | −7.57E−12 | 0.3705 | 0.2963 | 0.1304 | 0.3611 |

TABLE 4.2
Results for Pulse Wave Velocity from ECG-Distal GTEC sensor

| ECG-Dist | MEAN | STD | MAE | MSE | RMSE |
|---|---|---|---|---|---|
| GTEC | (m/s) | (m/s) | (m/s) | (m/s²) | (m/s) |
| Model 1 | −3.99330E−10 | 0.8202 | 0.6819 | 0.6391 | 0.7995 |
| Model 2 | −2.09E−13 | 0.3912 | 0.2908 | 0.1454 | 0.3813 |
| Model 3 | 3.28E−12 | 0.3713 | 0.2962 | 0.131 | 0.3619 |
| Model 4 | −2.37E−11 | 0.3703 | 0.2984 | 0.1303 | 0.361 |

TABLE 4.3
Results for Pulse Wave Velocity from Proximal-Distal GTEC sensors

| Prox-Dist | MEAN | STD | MAE | MSE | RMSE |
|---|---|---|---|---|---|
| GTEC | (m/s) | (m/s) | (m/s) | (m/s²) | (m/s) |
| Model 1 | −1.55E−12 | 0.6588 | 0.5334 | 0.4085 | 0.6391 |
| Model 2 | 2.13E−12 | 0.32 | 0.2667 | 0.0964 | 0.3105 |
| Model 3 | 9.24E−13 | 0.2936 | 0.2511 | 0.0811 | 0.2849 |
| Model 4 | −1.05E−11 | 0.2925 | 0.2531 | 0.0805 | 0.2838 |
| Model 5 | −5.01E−14 | 0.6208 | 0.4898 | 0.3627 | 0.6023 |
| Model 6 | −3.13E−12 | 0.4376 | 0.3402 | 0.1802 | 0.4245 |

A first noteworthy result regards the PWV estimates obtained from PTT (Table 4.3, Model 1 to Model 4) which are lower than those obtained from the PAT (Table 4.1 and 4.2). Secondly, better results can be achieved if other personal data of the subject under assessment are included in the linear regression model. The model that includes age, height and the heart rate median value indeed shows the best results.

Using the parameters of the PPG signal morphology does not lead to remarkable results. Apparently, the PWV estimate is not influenced by these PPG features.

The best method is, therefore, Method 4, formula shown in (1.9). FIG. 4.3 shows the PWV estimates compared to the gold standard measures, the Spearman correlation coefficient, and its p-value.

### 4. Blood Pressure

Blood Pressure estimates were obtained by linear regression of:
Method 1: PTT
Method 2: PTT+age+height+median(HR), formulas (1.12) and (1.13)
Method 3: PTT+Crest Time+Stiffness Index+Pulse Area from proximal sensor, formulas (1.14) and (1.15)
Method 4: PTT+Crest Time+Stiffness Index+Pulse Area from distal sensor

Results for systolic and diastolic blood pressure are presented separately in tables 5.1 and 5.2, respectively. The blood pressure is given in mmHg and so are its estimation errors.

TABLE 5.1
Results for Systolic Blood Pressure

| | MEAN | STD | MAE | MSE | RMSE |
|---|---|---|---|---|---|
| Systolic BP | (mmHg) | (mmHg) | (mmHg) | (mmHg²) | (mmHg) |
| Method 1 | 2.50E−08 | 10.5732 | 8.3936 | 105.2163 | 10.2575 |
| Method 2 | −0.0946 | 9.359 | 7.3368 | 82.447 | 9.08 |
| Method 3 | −0.5074 | 11.5253 | 8.9927 | 124.7884 | 11.1709 |
| Method 4 | −6.1145 | 18.1551 | 13.3295 | 346.3948 | 18.6117 |

TABLE 5.2
Results for Diastolic Blood Pressure

| | MEAN | STD | MAE | MSE | RMSE |
|---|---|---|---|---|---|
| Diastolic BP | (mmHg) | (mmHg) | (mmHg) | (mmHg²) | (mmHg) |
| Method 1 | −3.10E−08 | 9.3139 | 7.4511 | 81.6458 | 9.0358 |
| Method 2 | −0.052 | 9.1741 | 7.2174 | 79.2166 | 8.9004 |
| Method 3 | −0.3014 | 9.1693 | 7.179 | 78.9119 | 8.8832 |
| Method 4 | −0.727 | 11.8798 | 9.0639 | 132.8377 | 11.5255 |

Good estimates were obtained from method 2, since the error standard deviation is lower than the other methods for both systolic and diastolic blood pressure estimates.

In order to further reduce the error, a linear regression model with CT, SI, and PA was also tested.

In this case, the error standard deviation of systolic BP is greater than the one obtained with the other methods; on the other hand, the diastolic blood pressure estimates show an error standard deviation slightly lower than the other methods (Tables 4.14 and 4.15).

Regarding the systolic BP, the best method seems to be Method 2. For diastolic BP, Method 3 might be preferred. In FIGS. 3.4 and 3.5 estimated systolic BP and diastolic BP are presented, with Spearman's correlation coefficient and its p-value.

### 5. Heart Rate Variability HRV

Tables 5 and 6 show the results of Heart Rate Variability (HRV) analysis obtained through the proximal and distal PPG signals compared to the gold standard. 16 parameters were considered to estimate HRV as shown in tables 6.1 and 6.2:

TABLE 6.1
Results for HRV from Proximal E4 sensor

| Method | MEAN | STD | MAE | MSE | RMSE |
|---|---|---|---|---|---|
| min (IBI) [ms] | 1.7578 | 59.1934 | 26.3672 | 3.33E+03 | 57.7214 |
| max (IBI) [ms] | 5.2734 | 22.4316 | 10.7422 | 5.06E+02 | 22.4906 |
| median (IBI) [ms] | −0.1953 | 2.5268 | 1.5625 | 6.1035 | 2.4705 |
| mean (IBI) [ms] | −0.6009 | 1.8462 | 1.4547 | 3.599 | 1.8971 |
| min (HR) [bpm] | −0.5253 | 1.8986 | 0.8386 | 3.7003 | 1.9236 |
| max (HR) [bpm] | −0.2017 | 7.4056 | 3.2432 | 52.1419 | 7.2209 |
| median (HR) [bpm] | 0.0239 | 0.2131 | 0.1296 | 0.0437 | 0.2091 |
| mean (HR) [bpm] | 0.0597 | 0.1826 | 0.1367 | 0.0352 | 0.1877 |
| SDNN [ms] | 1.2789 | 3.4363 | 2.3019 | 12.8535 | 3.5852 |
| RMSSD [ms] | −1.9151 | 6.0547 | 4.0024 | 38.4942 | 6.2044 |
| NN50 [unitless] | 1.55 | 3.2359 | 2.55 | 12.35 | 3.5143 |
| pNN50 [%] | 1.3966 | 2.2145 | 1.9473 | 6.6096 | 2.5709 |
| SD1 [ms] | −7.2887 | 7.2565 | 9.095 | 103.1499 | 10.1563 |
| SD2 [ms] | 1.2984 | 2.7003 | 1.9866 | 8.6129 | 2.9348 |
| LF/HF [unitless] | −0.064 | 0.1309 | 0.0789 | 0.0204 | 0.1427 |
| Sample Entropy [unitless] | 0.3675 | 0.4653 | 0.3908 | 0.3407 | 0.5837 |

TABLE 6.2
Results for HRV from Distal E4 sensor

| Method | MEAN | STD | MAE | MSE | RMSE |
|---|---|---|---|---|---|
| min (IBI) [ms] | −9.5703 | 75.5377 | 38.0859 | 5.51E+03 | 74.2444 |
| max (IBI) [ms] | 1.1719 | 62.7577 | 25.7813 | 3.74E+03 | 61.1799 |
| median (IBI) [ms] | −9.9609 | 47.4281 | 12.5 | 2.24E+03 | 47.2882 |
| mean (IBI) [ms] | −8.6101 | 47.4905 | 13.1433 | 2.22E+03 | 47.082 |
| min (HR) [bpm] | −0.0625 | 5.1679 | 2.0708 | 2.54E+01 | 5.0375 |
| max (HR) [bpm] | 1.0942 | 8.9442 | 4.58 | 7.72E+01 | 8.7862 |
| median (HR) [bpm] | 0.938 | 4.5218 | 1.1889 | 2.03E+01 | 4.506 |
| mean (HR) [bpm] | 0.8257 | 4.5473 | 1.2591 | 2.03E+01 | 4.5084 |
| SDNN [ms] | 4.2712 | 11.9516 | 5.2593 | 153.9424 | 12.4074 |
| RMSSD [ms] | −1.0658 | 6.6971 | 4.3735 | 42.7448 | 6.614 |
| NN50 [unitless] | 5.7 | 13.9061 | 6.2 | 216.2 | 14.7037 |
| pNN50 [%] | 9.0508 | 20.9626 | 9.3783 | 499.3467 | 22.3467 |
| SD1 [ms] | 7.5588 | 16.0482 | 8.1973 | 301.8016 | 17.3724 |
| SD2 [ms] | −21.5641 | 30.2179 | 27.8905 | 1.33E+03 | 36.5031 |
| LF/HF [unitless] | −0.2491 | 0.6637 | 0.2555 | 0.4794 | 0.6924 |
| Sample Entropy [unitless] | 1.0458 | 1.0472 | 1.0458 | 2.1326 | 1.4604 |

Overall, estimates from the proximal PPG sensor are more accurate than those obtained from the distal sensor. This may be due to a more correct positioning of the wristband and a greater grip on the wrist.

Although its error standard deviation is relatively large, RMSSD can be considered as a valuable option for a future algorithm addressing the cardiovascular health of the subject wearing the PPG sensors.

The analysis of the cardiovascular parameter estimation has shown that there are multiple cardiovascular parameters that can be estimated with reasonable deviation from the reference. To conclude, the simple and low-cost PPG signal contains useful information about a person's cardiovascular health that lay far beyond the pulse rate, which is currently the most common extracted feature. The novel algorithms according to the present invention are capable of estimating cardiovascular parameters with only a slight deviation from the reference values even in case of two PPG sensors located at the wrist. This offers for the first time the possibility to include two PPG sensors within one wrist-worn device to provide a detailed analysis of the cardiovascular conditions of a subject. The two PPG sensors can be included into a fitness tracker or a smartwatch for permanent monitoring of those cardiovascular parameters.

FIG. 4 exemplarily shows a system 100 for determining cardiovascular parameters, namely vascular age index AgIx, blood pressure BPdia and BPsys, pulse wave velocity PWV, augmentation index Aix and heart rate variability HRV. The system 100 can be implemented in a wrist-worn device, such as a fitness tracker or a smartwatch and includes two PPG sensors 101, a processor 102, a memory 103, comparison with prestored data 104 and a user interface 105. The database 103 contains reference data for all cardiovascular parameters and may be derived from physiological data obtained from different organizations databases and obtained from measured data of the system 100. In another embodiment, a database can be externally coupled to the system through wired or wireless connectivity.

The PPG sensors 101 are configured to illuminate skin of a user and measure two PPG signals based on the illumination absorption by the skin. The PPG sensors 101 may include, for example, at least one periodic light source (e.g., light-emitting diode (LED), or any other periodic light source related thereof), and a photo detector configured to receive the periodic light emitted by the at least one periodic light source reflected from the user's skin. In a preferred embodiment, the PPG sensor comprises at least one green light source and comprises a sampling frequency of preferably 512 Hz.

The two PPG sensors 101 can be coupled to the processor 102. In another embodiment, the PPG sensors 101 may be included in a housing with the processor 102 and other circuit/hardware elements. It is preferred, when both PPG sensors 101 are included in a housing and are positioned with a distance of 5 cm or less, facing the dorsal part of the arm.

The processor 102 (for example, a hardware unit, an apparatus, a Central Processing Unit (CPU), a Graphics Processing Unit (GPU)) can be configured to receive and process the periodic light received from the PPG sensors 101. The processing includes pre-processing of the data at first instance as discussed before and estimation of the cardiovascular parameters with help of the algorithms according to the present invention. The estimated cardiovascular parameters are then compared with prestored data 104 and processed to the user interface 105 to be displayed for the user. The user can further provide feedback to the estimated parameters.

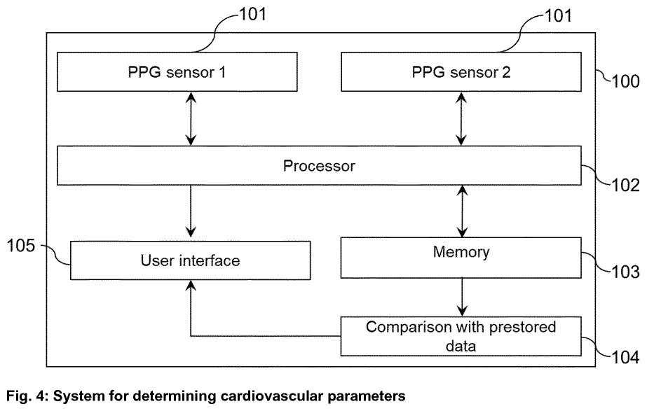

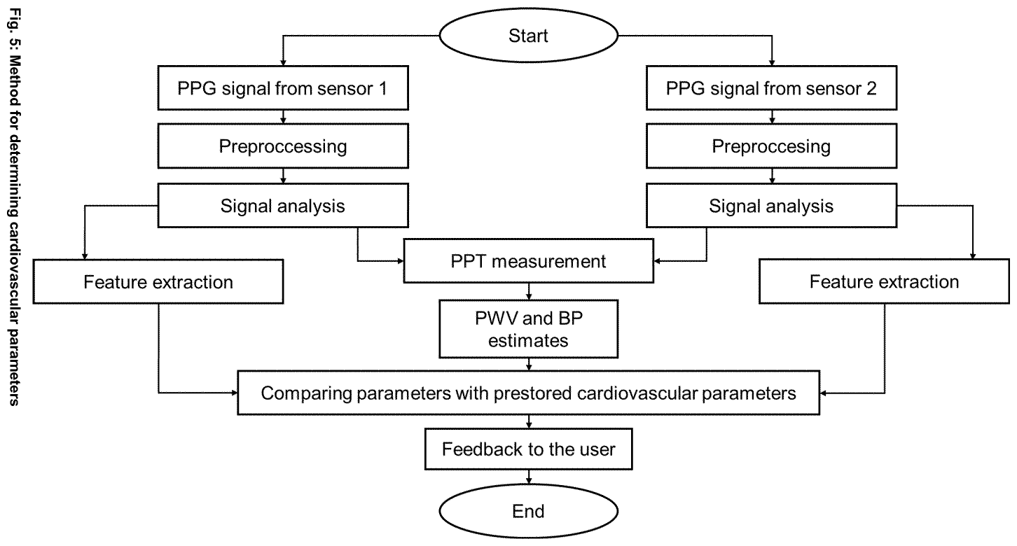

FIG. 5 is a flow diagram illustrating a method for estimating one or more cardiovascular parameters in a subject, according to an exemplary embodiment based on two PPG signals from two separate PPG sensors. Referring to FIG. 5, in operation, the electronic device illuminates skin of a user and measures the PPG signal from two PPG sensors based on the illumination absorption by the skin. For example, in the electronic device, as illustrated in FIG. 4, the two PPG sensors 101 are configured to illuminate the skin of the user and measure the PPG signal based on am illumination absorption by the skin.

In operation, the electronic device 100 extracts a plurality of parameters from both PPG signals, after preprocessing of the signal, including the PPG features, the HRV features, the APG features and the pulse transit time (PTT). Based on the two PPG signal analysis, the cardiovascular parameters can be estimated as described above. The electronic device 100 estimates the cardiovascular parameters, in this case PWV and BP based on the extracted plurality of parameters. The estimated parameters are compared with prestored cardiovascular parameters 104. The result is displayed within the user interface 105 giving feedback to the user.

With the help of the system and the method for estimating one or more cardiovascular parameters, the user can continuously monitor and evaluate physiological parameters, such as cardiovascular parameters. Based on the advanced algorithms including specific anatomical data, the evaluation of several cardiovascular parameters is achieved. The evaluation of supplementary parameters, such as blood flow, blood pressure, arterial stiffness, vessel elasticity, vascular age allows a comprehensive general health assessment. This individual cardiovascular health assessment reduces the risk of misinterpretation and leads to a more precise health assessment for the user.

1. A method for estimating one or more cardiovascular parameters in a subject having an age and a body height, the method comprising:
determining the age (p_{age}) and the body height (p_{height}) of the subject;
measuring at least two photoplethysmographic (PPG) signals with at least two PPG sensors at two different positions at the subject;
separating the PPG signal into PPG pulses, a start point and an end point of the pulse corresponding to a systolic foot of the PPG signal;
determining the heart rate of the subject (p_{HR}) and calculating the median heart rate;
determining systolic A_{sys} and diastolic A_{dia} peak amplitudes and their times tₛ and t_{d};
calculating the second derivative of the PPG pulse, and determining the characteristic points a, b, c, d, and e from the second derivative of the PPG pulse, a and e being a first and second most prominent maxima in the second derivative, respectively, c being a most prominent peak between the points a and e, b being a most prominent minimum in the second derivative, and d being a most prominent minimum between the points c and e,
determining
(a) a vascular age index AgIx using linear regression based on the characteristic points a, b, c, d, and e, age (p_{age}), body height (p_{height}), and median heart rate of the subject,
(b) a pulse wave velocity PWV using linear regression based on a time difference between the two PPG pulses (PTT), age (p_{age}), body height (p_{height}), and median heart rate estimation of the subject,
(c) blood pressure BP_{dia} and BP_{sys} using linear regression based on the time difference between the two PPG pulses (PTT) and median heart rate, and
(d) optionally an augmentation index AIx, based on the systolic A_{sys} and diastolic A_{dia} peak amplitudes normalized to 75 heartbeats (AIx@75) and using a linear regression based on the normalized augmentation index AIx; and
outputting calculated parameters.

2. The method of claim 1, further comprising:
determining Crest Time (CT), Stiffness Index (SI), and Pulse Area (PA) of the PPG signal,
wherein the cardiovascular parameters are estimated with equations:
(a) vascular age index AgIx:
AgIx=d₀+d₁ [symbol] x+d₂p_{age}+d₃p_{height}+d₄me [symbol] R),
wherein [symbol] is estimated based on characteristic points a, b, c, d, and e:
$$= 45.4 * \frac{b - c - d - e}{a} + 6 \; 5.9 ;$$
(b) pulse wave velocity PWV:
PWV=g₀+g₁ [symbol] +g₂p_{age}+g₃p_{height}+g₄ [symbol] R);
(c) blood pressure BPdia and BPsys:
BP_{dia}=l_{0d}+l_{1d} [symbol] +l_{2d} [symbol] R)+l_{3d}CTₚ+l_{4d}SIₚ+l_{5d}PAₚ
BP_{sys}=k₀ₛ+k₁ₛ [symbol] +k₂ₛp_{age}+k₃ₛp_{height}+k₄ₛ [symbol] R);
(d) normalized augmentation index AIx@75: [symbol]
=(x−y)/y by the sum of two exponential, and
AIx@75=b₀+b₁A [symbol] 5,
wherein AIx@75 is an augmentation index (AIx) normalized to 75 heartbeats,
wherein, p_{age} is the age and p_{height} is the body height of the subject, median (HR) is the median heart rate, PTT is the time difference between the PPG pulses, A_{sys} and A_{dia} are magnitudes of the systolic and diastolic peak, respectively, CT is the Crest Time, ST is the Stiffness Index and PA is the Pulse Area of the PPG signal, x is the diastolic peak amplitude and y is the systolic peak amplitude and d₀ to d₄, g₀ to g₄, l_{0d} to l_{5d}, k₀ₛ to k₄ₛ, and b₀ to b₁ are coefficients of respective linear regression equations.

3. The method of claim 1, wherein first and second PPG sensors are located on a wrist of the subject, with a distance of 5 cm or less between the first and second PPG sensors.

4. The method of claim 1, wherein the cardiovascular parameters are estimated based on at least 60 PPG pulses.

5. The method of claim 1, further comprising determining heart rate variability, HRV, by calculating one or more of:
a minimum and maximum interbeat interval (IBI);
a median and mean IBI;
a minimum and maximum heart rate;
a median and mean heart rate;
a standard deviation of the IBI of normal sinus beats (SDNN);
a number of adjacent intervals that differ from each other by more than 50 ms (NN50 and pNN50);
a root mean square of successive difference between normal heartbeats (RMSSD);
a LF/HF ratio between a low-frequency power, in a range of from 0.04 to 0.15 Hz, and the high-frequency power in a range of from 0.15 to 0.4 Hz;
a standard deviation (SD1) of a distance of each point from the x-axis in a Poincaré Plot, obtained by plotting every IBI interval against the prior interval;
a standard deviation (SD2) of each point from the y=x+mean (IBI interval) in a Poincaré Plot, obtained by plotting every IBI interval against the prior interval; and
a sample entropy.

6. The method of claim 1, wherein the characteristic points a, b, c, d, and e are automatically derived from the second derivative of the PPG pulse, and
wherein
a and e are the first and second most prominent maxima in the second derivative, respectively,
c is the most prominent peak between the points a and e,
b is the most prominent minimum in the second derivative, and
d is the most prominent minimum between points c and e.

7. The method of claim 1, wherein the systolic A_{sys} and diastolic A_{dia} peak amplitudes and their times tₛ and t_{d} are determined by:
modeling the PPG waveform as a sum of two pulse waves through exponential functions and applying nonlinear regression to fit the model to the PPG waveform and receive estimates of tₛ and t_{d} to find A_{sys} and A_{dia}, respectively, or
modeling the first wave with known position at the systolic peak A_{sys}, and subtracting its exponential model from the PPG signal and thereby yielding the remaining reflected wave.

8. The method of claim 1, wherein the one or more calculated parameters are displayed on a human body health monitoring device comprising a first and a second PPG sensor.

9. The method of claim 1, additionally outputting an acoustic or visual signal together with the calculated parameter.

10. The method of claim 1, further comprising:
comparing the calculated cardiovascular parameters with prestored cardiovascular index parameters; and
outputting an acoustic or visual signal, if the calculated cardiovascular parameters differ more than X % from the prestored cardiovascular index parameters, whereas X is 5, 10, 20, 30, 40, 50, 60, 70, 80, 90, or 100.

11. A wrist-worn device configured for determining one or more of:
a vascular age index, AgIx;
a pulse wave velocity, PWV;
a blood pressure, BP_{dia} and BP_{sys};
augmentation index, AIx,
wherein the device comprises
first and second PPG sensors, separated by a distance of 5 cm or less, facing a dorsal part of the arm, the PPG sensor comprising a green light source with a sampling frequency.

12. The device of claim 11, further comprising a signal processor adapted to calculate one or more of:
vascular age index, AgIx, using linear regression based on the characteristic points a, b, c, d, and e, age (p_{age}), body height (p_{height}) and median heart rate of the subject;
pulse wave velocity, PWV, using linear regression based on a time difference between two PPG pulses (PTT), the age (p_{age}), the body height (p_{height}) and the median heart rate estimation of the subject;
blood pressure, BP_{dia} and BP_{sys}, using linear regression based on the time difference between the two PPG pulses (PTT) and median heart rate; and
optionally, an augmentation index, AIx, based on systolic A_{sys} and diastolic A_{dia} peak amplitudes normalized to 75 heartbeats (AIx@75) and using a linear regression based on a normalized augmentation index, AIx.

13. The device of claim 11, wherein the sampling frequency is 512 Hz.

14. The method of claim 1, wherein the cardiovascular parameters are estimated based on at least 100 PPG pulses.

15. The method of claim 1, wherein the cardiovascular parameters are estimated based on at least 120 PPG pulses.
